(19) United States
(12) Patent Application Publication
(10) Pub. No.: US 2022/0054009 A1
(43) Pub. Date: Feb. 24, 2022

(54) INSTRUMENT FOR IMAGING THE ORAL CAVITY

(71) Applicant: Denti-Flex Cam, LLC, Phoenix, AZ (US)

(72) Inventors: Wayne Friedling, Fountain Hills, AZ (US); Edward Gomez, Phoenix, AZ (US); Laura Gomez, Phoenix, AZ (US); Ashley Delgado, Phoenix, AZ (US)

(73) Assignee: Denti-Flex Cam, LLC, Phoenix, AZ (US)

(21) Appl. No.: 16/998,785

(22) Filed: Aug. 20, 2020

(51) Int. Cl. A61B 5/00

(52) U.S. Cl. CPC A61B 5/004; A61B 5/002; A61B 5/4542; A61B 5/0077

(57) ABSTRACT

Disclosed is a device for use in the oral cavity that includes a (1) handle, (2) camera, scanner, or other imaging device (collectively, “camera”), and (3) flexible stem having a first end attached to the handle and a second end attached to the camera. The stem can be moved by an operator into a bent (or curved) position suitable for taking images in various locations in the oral cavity, such as in the back of the mouth and/or behind the teeth. The stem requires an appropriate amount of bending force to be moved into a curved position and then out of the curved position into another position. The flexible stem is configured to require enough bending force so that it does not relax sufficiently to move out of position during normal use.

### BACKGROUND

Dentists or clinicians use an instrument (or device) to image the oral cavity, particularly the teeth, in order to have an accurate visual image of the inside of the mouth. Such devices typically have a camera at the end of a handle. Problems with known devices are that they are too short and/or rigid to easily reach into the rear of the mouth and/or behind the teeth. So, it is difficult to properly position the device to obtain the proper images.

### SUMMARY OF THE INVENTION

Disclosed is an instrument for use in the oral cavity. The instrument includes a (1) handle, (2) camera, scanner, or other imaging device (collectively, “camera”), and (3) a flexible stem having a first end attached to the handle and a second end that is attached to the camera. The camera can be of any type suitable for taking still or video images in the oral cavity. The stem can be moved by an operator into a bent (or curved) position suitable for taking images in various locations in the oral cavity, such as in the back of the mouth and/or behind the teeth. The stem requires an appropriate amount of bending force to be moved into a curved position and then out of the curved position and into another position. The stem is configured to require enough bending force so that it does not relax sufficiently to move out of position during normal use—force must be applied to change its position. The stem has a suitable length and diameter to be used in the oral cavity, and the handle is configured so that operator can easily hold, maneuver, and operate the device.

### BRIEF DESCRIPTION OF THE DRAWINGS

FIG. 1 is a side view of a device in accordance with this disclosure in a straight position.

FIG. 2 is an alternate side view of the device of FIG. 1 with the stem in a curved position.

FIG. 3 is a side, perspective view of the device of FIG. 2.

FIG. 4 is bottom view of the device of FIG. 2.

FIG. 5 is an alternate side, perspective view of the device of FIG. 2.

FIG. 6 is a cross-sectional view taken along line A-A of FIG. 1.

FIG. 7 is a cross-sectional view of the stem taken along line B-B of FIG. 3.

### DETAILED DESCRIPTION OF EMBODIMENTS

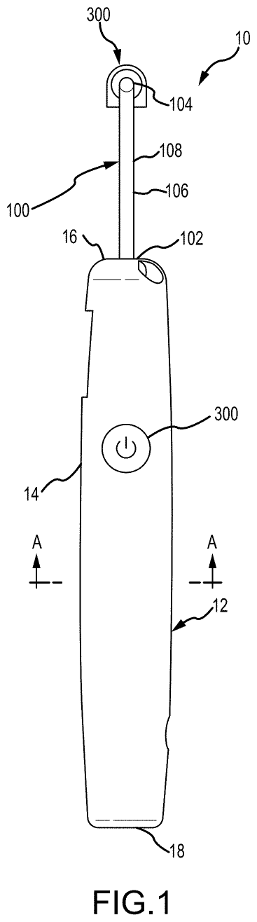

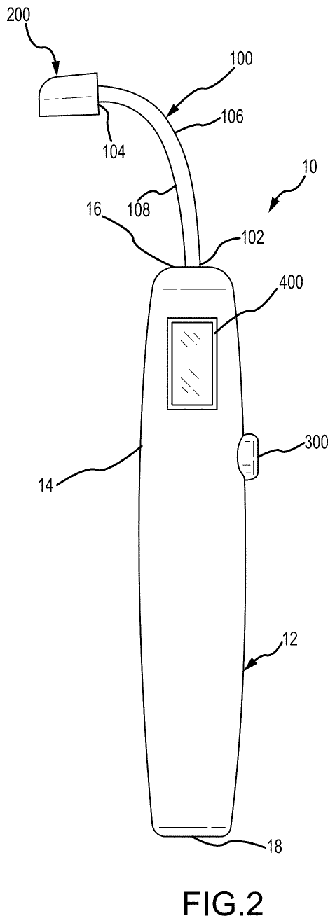

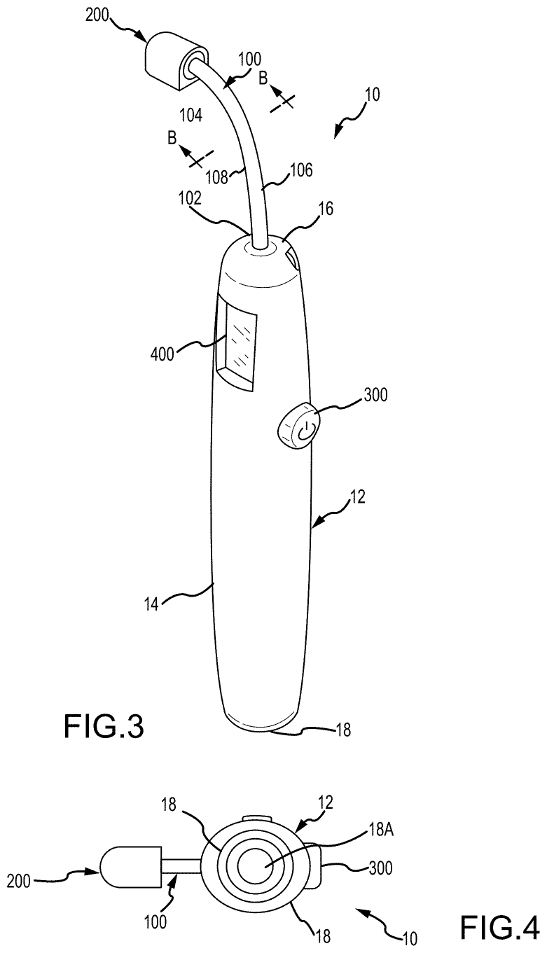

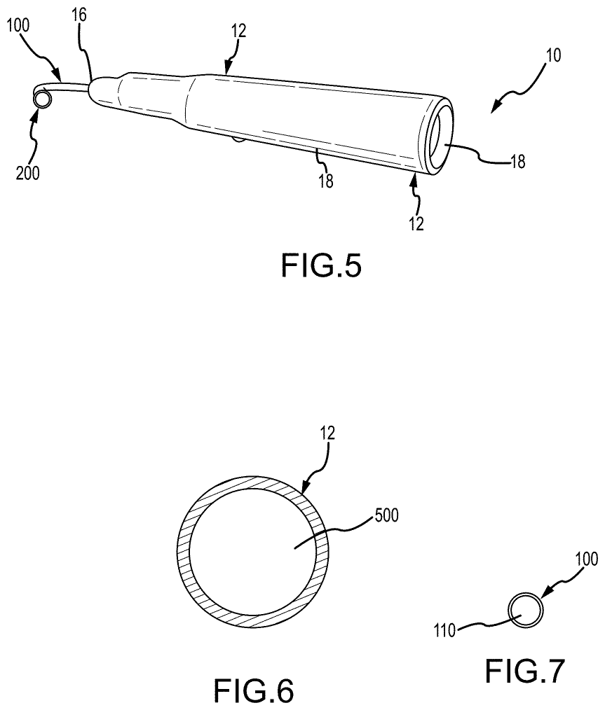

Turning now to the figures for the purpose of describing embodiments of the invention and not to limit the scope of the claims, FIGS. 1-7 show an embodiment of a device according to this disclosure.

Device 10 comprises a handle 12, a flexible stem 100, a camera 200, an optional control 300, and an optional display 400. In a preferred embodiment, handle 12 is comprised of any suitable material, such as plastic or metal. Handle 12 has a generally, smooth, curved outer surface 14 that is configured to be held easily in a user's (or operator's) hand. As shown, handle 12 has a proximal (or first) end 16, and a distal (or second) end 18. An induction charging port 18A may be at second end 18.

In this embodiment, handle 12 tapers inwards at first end 16 and tapers inwards at second end 18. Handle 12 may have an outer diameter of any amount from about ½″-2″ where it juxtaposes first end 16, an outer diameter of any amount from about ½″-2.5″ at its center, and an outer diameter of any amount from about ½″-2″ where it juxtaposes second end 18. However, handle 12 may be of any suitable size and shape.

Handle 12 preferably has a length as measured from first end 16 to second end 18 of any amount from about: 3″-10″, or about 5″-10″, or about 6″-9″, or about 6″-8″, or about 4″-7″, although any suitable length may be used. The distance from first end 16 to the center of control 300 is preferably any amount from about 1″-6.″ The distance from first end 16 to the center of display 400 is preferably any amount from about 1″-6″.

Handle 12 preferably has a cavity 500, best seen in FIG. 6, that can retain electronics for storing and for transmitting images taken by camera 200. In one embodiment, cavity 500 retains a PCB (not shown) that includes a memory, a processor, and a transmitter. Alternatively, handle 12 need only contain a transmitter if it does not store, organize, or analyze images or video received by camera 200, or camera 200 can itself include a transmitter and send images it takes. In that case, the camera 200 may communicate directly with a computing device, in which case no wires or electronics in handle 12 or stem 100 would be required.

Handle 12 may include a power source, such as a battery, and/or be hard wired to an AC or DC power source. If handle 12 includes a transmitter, the transmitter may communicate using any suitable protocol, such as WIFI, RF, Blue Tooth or cellular network, directly or indirectly with a remote electronic device such as a server, computer or cell phone. A connection between device 10 and a power source could be made by a USB or any suitable connector, such as induction charging port 18A. Handle 12 may also be hard-wired to a computing device and include one or more wires or fiber optics (not shown) that directly or indirectly connects the computing device to the camera.

The PCB, if included, is of any suitable shape, size, and configuration and formed of any suitable material, and includes suitable circuitry, for receiving, storing, analyzing, and/or storing information from camera 200.

Stem 100 is flexible, is preferably hollow with an inner lumen 110 (shown in FIG. 7), and is preferably comprised of metal or plastic. Stem 100 can be bent by a user into a curved position suitable to take pictures or videos of the oral cavity. Stem 100 has a proximal (or first) end 102, a distal (or second) end 104, and a body portion 106, which as shown is cylindrical with an outer surface 108. The second end 104 is configured to connect to camera 200 in any suitable manner, and camera 200 may have a hinged or swivel-type connection to second end 104 so it can be moved separately from stem 100. Alternatively, the connection can be fixed so camera 200 cannot move independently of stem 100.

Stem 100 is preferably any suitable length, as measured from first end 102 to second end 104, from about 4″-10″, or about 3″-6″, or about 2″-6″, or about 2.5″-10″. Stem 100 has an outer diameter of any suitable amount from about ⅛″-½″, or about ¼″-⅝″, or about ⅜″-¾″ (and most preferably has a diameter less than the diameter of handle 12).

One aspect of the stem 100 is that it can be bent by an operator into a position suitable for photographing or taking a video of the oral cavity, and it remains in the bent (or curved) position until an appropriate amount of force is used to bend it back to its original position or to another position. Stem 100 can be moved into a position in which it has a curve having a radius of about ½″ or greater, about 1″ or greater, or about 1.5″ or greater, or about 2″ or greater, or about 2.5″ or greater, or abut 3″ or greater, or to any radius from ½″-3.5″, by applying a force to the second end 104. The force required to be applied to second end 104 of stem 100 to move it into one of the curved positions could be about: 3 lbs. or more, 3.5 lbs. or more, 4 lbs. or more, 5 lbs. or more, 5.5 lbs. or more, 6 lbs. or more, 6.5 lbs. or more, 7 lbs. or more, 7.5 lbs. or more, 8 lbs. or more, 8.5 lbs. or more, 9 lbs. or more, 9.5 lbs. or more, 10 lbs. or more, 10.5 lbs. or more, 11lbs. or more, 11.5 lbs. or more, 12 lbs. or more, 12.5 lbs. or more, 13 lbs. or more, 13.5 lbs. or more, 14 lbs. or more, 14.5 lbs. or more, 15 lbs. or more, 15.5 lbs. or more, 16 lbs. or more, 16.5 lbs. or more, 17 lbs. or more, 17.5 lbs. or more, 18 lbs. or more, 18.5 lbs. or more, 19 lbs. or more, 19.5 lbs. or more, 20 lbs. or more, or any amount from 2.5 lbs. to 25 lbs.

Once moved to a curved position, the stem 100 can be moved to a straight position or to another curved position by applying sufficient force, and preferably at least one of the forces recited in the preceding paragraph. That way, the stem remains in the curved position desired by the operator while in use.

In another embodiment, the stem 100 is 4″-6″ in length and can be bent into a curve having a radius of 2″ by applying five pounds or more of force.

In another embodiment, the stem 100 can be moved into a position in which it has a radius having a diameter of 1″ by applying eight pounds of force or more, and can also be moved into a position in which it has a radius of ¾″ by applying a force of 10 pounds or more.

One preferred camera 200 has a lens angle of between 60 mm and 135 mm. One type of camera 200 that may be used is a world view camera, 60 FPS, HD, wide angle. The visual data sensed by the camera 200 is communicated to the PCB (or directly to an external computer, server or mobile phone) by a wired or a wireless connection.

The lens of camera 200 may be formed of any suitable material, such as glass or plastic, and may be clear, colored any suitable color, include metallic or metal-oxide particles, and/or have any suitable coating.

Control 300 as shown extends outward from handle 12 by about 1/32″-¼″, is circular and has a diameter of about 3/16″-½″, although any suitable size and shape control 300 may be used. Control 300 can be used to operate camera 200, send images, or for any suitable function.

Display 400 is optional and, as shown, is a plastic window that is any amount from about ⅜″-1″ in width and any amount from about ½″-3″ in length. Display 400 can be configured to show any suitable information to an operator.

Some non-limiting examples of this disclosure follow:

Example 1: A device comprising:

a handle;

a stem having a length from 3″-10″, a diameter from ⅛″-½″, and configured to be moved from a straight position into a curved position in which it has a curve having a diameter of 1″ by applying five pounds or more of force to a second end of the stem, wherein the stem further comprises a first end connected to the handle; and

a camera connected to the second end of the stem.

Example 2: The device of example 1 that further includes a storage medium to store data received by the camera.

Example 3: The device of examples 1 or 2, wherein the stem has an inner lumen and that further includes circuitry in the inner lumen, wherein the circuitry connects to the camera.

Example 4: The device of example 3, wherein the circuitry is one or more wires or fiber optic lines.

Example 5: The device of example 3 that further includes a storage medium to store data received by the camera, and the circuitry connects to the storage medium.

Example 6: The device of any of examples 1-5, wherein the handle is comprised of plastic or metal.

Example 7: The device of any of examples 1-6, wherein the stem is comprised of plastic or metal.

Example 8: The device of any of examples 1-7, wherein the stem is comprised of stainless steel.

Example 9: The device of any of examples 1-8, wherein a camera lens is exposed at the second end of the stem.

Example 10: The device of example 9, wherein the camera lens has a diameter that is equal to or less than a diameter of the flexible stem.

Example 11: The device of any of examples 1-10, wherein the handle has a length from 3″-10″.

Example 12: The device of any of examples 1-11, wherein the flexible stem has horizontal ribs.

Example 13: The device of any of examples 1-12, wherein the stem is 3″-10″ in length and can be bent into a curve having a diameter of 2″ by applying five pounds or more of force.

Example 14: The device of any of examples 1-13, wherein the stem can be moved back to its first position by applying five pounds or more of force.

Example 15: The device of any of examples 1-14, wherein the stem can be moved into a position in which it has a curve having a diameter of 2″ or more by applying five pounds or more of force, can be moved into a position in which it has a curve having a diameter of 1″ by applying eight pounds of force or more, and can be moved into a position in which it has a curve with a diameter of ¾″ by applying 10 pounds or more of force to the second end of the stem.

Example 16: The device of any of examples 1-15, wherein the camera lens has a diameter that is greater than the diameter of the flexible stem.

Example 17: The device of any of examples 1-16 that further includes a communication device that operates on one or more of the following protocols: ZiBi, Bluetooth, Wi-Fi, or a wired connection.

Example 18: The device of any of examples 1-17, wherein the camera has a lens that is convex.

Example 19: The device of any of examples 1-18, wherein the camera had a lens with a 120° or greater view outward from the lens position.

Example 20: The device of any of examples 1-19, wherein the handle has a length that is greater than the length of the flexible stem.

Example 21: The device of any of examples 1-20, wherein the stem is configured to be moved to a position other than the curved position by applying 5 lbs. or more of force to the second end of the stem.

Example 22: The device of any of examples 1-21, wherein the force is greater than 5 lbs. to move the stem to the curved position.

Example 23: The device of example 21 or 22, wherein the force is greater than 5 lbs. to move the stem from the curved position to another position.

Example 24: The device of any of examples 1-21 or 23, wherein the force to move the stem to the curved position is about: 5.5 lbs. or more, 6 lbs. or more, 7 lbs. or more, 8 lbs. or more, 9 lbs. or more, 10 lbs. or more, 11 lbs. or more, 12 lbs. or more, 13 lbs. or more, 4 lbs. or more, 15 lbs. or more, or any amount from about 5 lbs. to about 20 lbs.

Example 25: The device of any of examples 21-25, wherein the force required to move the stem from the curved position to another position is about: 5.5 lbs. or more, 6 lbs. or more, 7 lbs. or more, 8 lbs. or more, 9 lbs. or more, 10 lbs. or more, 11 lbs. or more, 12lbs. or more, 13 lbs. or more, 4 lbs. or more, 15 lbs. or more, or any amount from about 5 lbs. to about 20 lbs.

Example 26: A device comprising:

a handle;

a stem having a length from 3″-10″, a diameter from ⅛″-½″, and configured to be moved from a straight position into a curved position in which it has a curve having a diameter of 1″ by applying from 2.5 lbs. to 25 lbs. of force to a second end of the stem, wherein the stem further comprising a first end connected to the handle; and

a camera connected to the second end of the stem.

Having thus described some embodiments of the invention, other variations and embodiments that do not depart from the spirit of the invention will become apparent to those skilled in the art. The scope of the present invention is thus not limited to any particular embodiment, but is instead set forth in the appended claims and the legal equivalents thereof. Unless expressly stated in the written description or claims, the steps of any method recited in the claims may be performed in any order capable of yielding the desired result.

What is claimed is:

1. A device having:
(a) a handle;
(b) a stem having a first end connected to the handle, a second end, length as measured between the first end and the second end from 3″-10″, a diameter from ⅛″-½″, and configured to be moved from a straight position into a position in which it has a curve having a diameter of 1″ by using five pounds or more of force to the second end, wherein the flexible stem has a first end connected to the handle and a second end; and
(c) a camera connected to the second end of the stem.

2. The device of claim 1 that further includes a storage medium in the handle to store data received by the camera.

3. The device of claim 1, wherein the stem has an inner lumen and that further includes circuitry in the inner lumen, wherein the circuitry connects to the camera.

4. The device of claim 3, wherein the circuitry is one or more wires or fiber optic lines.

5. The device of claim 3 that further includes a storage medium in the handle to store data received by the camera, and the circuitry connects to the storage medium.

6. The device of claim 1, wherein the handle is comprised of plastic or metal.

7. The device of claim 1, wherein the stem is comprised of plastic or metal.

8. The device of claim 1, wherein the stem is comprised of stainless steel.

9. The device of claim 1, wherein a camera lens is exposed at the second end of the stem.

10. The device of claim 9, wherein the camera lens has a diameter that is equal to or less than a diameter of the flexible stem.

11. The device of claim 1, wherein the handle has a length from 3″-8″.

12. The device of claim 1, wherein the stem has horizontal ribs.

13. The device of claim 1, wherein the stem is 3″-10″ in length and can be bent into a curve having a diameter of 2″ by applying five pounds or more of force.

14. The device of claim 1, wherein the stem can be moved back to its first position by applying five pounds or more of force.

15. The device of claim 1, wherein the stem can be moved into a position in which it has a curve having a diameter of 2″ or more by applying five pounds or more of force, can be moved into a position in which it has a curve having a diameter of 1″ by applying eight pounds of force or more, and can be moved into a position in which it has a curve with a diameter of ¾″ by applying a force of 10 pounds or more.

16. The device of claim 1, wherein the camera lens has a diameter that is greater than the diameter of the flexible stem.

17. The device of claim 1 that further includes a communication device that operates on one or more of the following protocols: ZiBi, Bluetooth, Wi-Fi, or a wired connection.

18. The device of claim 1, wherein the camera had a lens with a 120° or greater view outward from the lens position.

19. The device of claim 1, wherein the handle has a length that is greater than a length of the stem.

20. The device of claim 1, wherein the stem has a length that is greater than a length of the handle.
